(19) United States
(12) Patent Application Publication
(10) Pub. No.: US 2024/0041937 A1
(43) Pub. Date: Feb. 8, 2024

(54) PROPHYLAXIS AND TREATMENT OF ORTHOPOX VIRUSES USING REGENERATIVE CELLS AND PRODUCTS THEREOF

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/366,527

(22) Filed: Aug. 7, 2023

Related U.S. Application Data
(60) Provisional application No. 63/395,839, filed on Aug. 7, 2022.

(51) Int. Cl. A61K 35/28; C12N 5/0775; C12N 5/078; C12N 5/0789

(52) U.S. Cl. CPC A61K 35/28; C12N 5/0662; C12N 5/0644; C12N 5/0647; C12N 5/065

(57) ABSTRACT

Disclosed are prophylactic and therapeutic approaches to Orthopox viral infections. In one embodiment the invention teaches utilization of regenerative cell conditioned media as a prophylactic/therapeutic agent. Synergies with antivirals and immunotherapies are further described. In one specific embodiment, mesenchymal stem cells are activated in vitro with trigger agents activating Toll-Like Receptors (TLRs), NOD-Like Receptors (NLRs), and RIG-I-Like Receptors (RLRs) and conditioned media is isolated and utilized as a therapeutic. Quantification of activity is performed by assessment of antiviral activity and/or ability to stimulate NK mediated cytolysis. Additionally, means of treating Orthopox viral infections (Smallpox, Monkeypox, etc.) by direct administration of stem cells and/or products thereof such as exosomes are disclosed.

### CROSS-REFERENCE TO RELATED APPLICATIONS

This application claims priority to U.S. Provisional Application No. 63/395,839, titled “Prophylaxis and Treatment of Orthopox Viruses Using Regenerative Cells and Products Thereof” and filed Aug. 7, 2022, which is hereby incorporated by reference in its entirety.

### FIELD OF THE INVENTION

The invention is directed to methods of treating and preventing Orthopox viruses using regenerative cells.

### BACKGROUND OF THE INVENTION

Human monkeypox (MPX) is a rare zoonotic infection characterized by smallpox-like signs and symptoms. It is caused by monkeypox virus (MPXV), a double stranded DNA virus belonging to the genus Orthopox virus. MPX was first identified in 1970 and mostly prevailed in the rural rainforests of Central and West Africa in the past. Outside Africa, MPX was reported in the United Kingdom, the USA, Israel, and Singapore. In 2022, the resurgence of MPX in Europe and elsewhere posed a potential threat to humans. MPXV was transmitted by the animals-human or human-human pathway, and the symptoms of MPXV infection are similar to that of smallpox, but in a milder form and with lower mortality (1% to 10%). Although the smallpox vaccination has been shown to provide 85% protection against MPXV infection, and two anti-smallpox virus drugs have been approved to treat MPXV, there are still no specific vaccines and drugs against MPXV infection.

Therefore, there is an urgent need in the art to take active measures including the adoption of novel anti-MPXV strategies to control the spread of MPXV and prevent MPX epidemic. In this review, we summarize the biological features, epidemiology, pathogenicity, laboratory diagnosis, and prevention and treatment strategies on MPXV.

### SUMMARY

Preferred embodiments are directed to methods of prophylaxis and/or treatment of Orthopox Viruses (Monkeypox, Smallpox, etc) comprising: a) obtaining a regenerative cell population; b) stimulating said regenerative cell population with a ligand for Toll-Like Receptors (TLRs) and/or NOD-Like Receptors (NLRs), and/or RIG-I-Like Receptors (RLRs); c) collecting supernatant from said stimulated cells; d) concentrating active ingredients from said supernatant; and e) administered said concentrated supernatant.

Preferred methods include embodiments wherein said cellular population is a mesenchymal stem cell (MSC).

Preferred methods include embodiments wherein said MSC is derived from a source selected from a group comprising of: a) peripheral blood; b) bone marrow; c) placenta; d) umbilical cord blood; e) menstrual blood; f) amniotic fluid; g) cerebral spinal fluid; h) umbilical cord tissue; i) liver: j) spleen; k) kidney; l) skin; or m) adipose tissue.

Preferred methods include embodiments wherein said MSC express CD90.

Preferred methods include embodiments wherein said MSC express CD105.

Preferred methods include embodiments wherein said MSC express CD73.

Preferred methods include embodiments wherein said MSC express CD36.

Preferred methods include embodiments wherein said MSC express VEGF-receptor 2.

Preferred methods include embodiments wherein said MSC express CD115.

Preferred methods include embodiments wherein said MSC express c-met.

Preferred methods include embodiments wherein said MSC express interleukin-3 receptor.

Preferred methods include embodiments wherein said MSC express IGF receptor.

Preferred methods include embodiments wherein said MSC express EGF receptor.

Preferred methods include embodiments wherein said MSC express LDL receptor.

Preferred methods include embodiments wherein said cellular population is monocytes.

Preferred methods include embodiments wherein said monocytes are type 2 monocytes.

Preferred methods include embodiments wherein said cellular population is thymic medullary epithelial cells.

Preferred methods include embodiments wherein said cellular population is hematopoietic stem cells.

Preferred methods include embodiments wherein said hematopoietic stem cells express CD34.

Preferred methods include embodiments wherein said hematopoietic stem cells express CD133.

Preferred methods include embodiments wherein said hematopoietic stem cells possess ability to generate lymphoid cells when administered into an immune deficient mouse.

Preferred methods include embodiments wherein said hematopoietic stem cells possess ability to generate myeloid cells when administered into an immune deficient mouse.

Preferred methods include embodiments wherein said hematopoietic stem cells possess ability to generate erythroid cells when administered into an immune deficient mouse.

Preferred methods include embodiments wherein said hematopoietic stem cells possess ability to generate megakaryocytic cells when administered into an immune deficient mouse.

Preferred methods include embodiments wherein said hematopoietic stem cells possess ability to generate megakaryocytic cells when administered into an immune deficient mouse.

Preferred methods include embodiments wherein said cellular population is a mesenchymal stem cell.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are derived from the bone marrow.

Preferred methods include embodiments wherein said tissue derived mesenchymal stem cells are derived from perivascular tissue.

Preferred methods include embodiments wherein said tissue derived mesenchymal stem cells are derived from adipose tissue.

Preferred methods include embodiments wherein said tissue derived mesenchymal stem cells are derived from placental tissue.

Preferred methods include embodiments wherein said tissue derived mesenchymal stem cells are derived from amniotic tissue.

Preferred methods include embodiments wherein said tissue derived mesenchymal stem cells are derived from omental tissue.

Preferred methods include embodiments wherein said tissue derived mesenchymal stem cells are derived from deciduous tooth tissue.

Preferred methods include embodiments wherein said tissue derived mesenchymal stem cells are derived from umbilical cord tissue.

Preferred methods include embodiments wherein said tissue derived mesenchymal stem cells are derived from fallopian tube tissue.

Preferred methods include embodiments wherein said tissue derived mesenchymal stem cells are derived from hepatic tissue.

Preferred methods include embodiments wherein said tissue derived mesenchymal stem cells are derived from renal tissue.

Preferred methods include embodiments wherein said tissue derived mesenchymal stem cells are derived from cardiac tissue.

Preferred methods include embodiments wherein said tissue derived mesenchymal stem cells are derived from tonsillar tissue.

Preferred methods include embodiments wherein said tissue derived mesenchymal stem cells are derived from ovarian tissue.

Preferred methods include embodiments wherein said tissue derived mesenchymal stem cells are derived from neuronal tissue.

Preferred methods include embodiments wherein said tissue derived mesenchymal stem cells are derived from auricular tissue.

Preferred methods include embodiments wherein said tissue derived mesenchymal stem cells are derived from colonic tissue.

Preferred methods include embodiments wherein said tissue derived mesenchymal stem cells are derived from submucosal tissue.

Preferred methods include embodiments wherein said tissue derived mesenchymal stem cells are derived from hair follicle tissue.

Preferred methods include embodiments wherein said tissue derived mesenchymal stem cells are derived from hair pancreatic tissue.

Preferred methods include embodiments wherein said tissue derived mesenchymal stem cells are derived from hair muscle tissue.

Preferred methods include embodiments wherein said tissue derived mesenchymal stem cells are derived from hair subepithelial umbilical cord tissue.

Preferred methods include embodiments wherein said tissue derived mesenchymal stem cells are isolated from tissues containing cells selected from a group of cells comprising of: mesenchymal cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, and salivary gland serous cells.

Preferred methods include embodiments wherein said dedifferentiation is accomplished by introduction into cells proteins capable of inducing dedifferentiation.

Preferred methods include embodiments wherein said dedifferentiation results in cells expression pluripotency markers.

Preferred methods include embodiments wherein said pluripotency marker is TRA-1-60

Preferred methods include embodiments wherein said proteins capable of inducing dedifferentiation are selected from a group comprising of: a) OCT4; b) NANOG; c) KLF-1; d) SOX-2; and e) h-RAS.

Preferred methods include embodiments wherein mRNA is introduced into said cells in order to induce expression of pluripotency inducing genes.

Preferred methods include embodiments wherein said dedifferentiated cells are capable of proliferating for more than 50 passages.

Preferred methods include embodiments wherein said MSC are activated with a mimic of an injury signal to endow enhanced growth factor production from said MSC.

Preferred methods include embodiments wherein said mimic of an injury signal is lipopolysaccharide.

Preferred methods include embodiments wherein said mimic of an injury signal is poly (IC).

Preferred methods include embodiments wherein said mimic of an injury signal is free histones.

Preferred methods include embodiments wherein said mimic of an injury signal is oxytocin.

Preferred methods include embodiments wherein said mimic of an injury signal is flagellin.

Preferred methods include embodiments wherein said mimic of an injury signal is a heat shock protein.

Preferred methods include embodiments wherein said mimic of an injury signal is hsp90.

Preferred methods include embodiments wherein said mimic of an injury signal is hsp60.

Preferred methods include embodiments wherein said mimic of an injury signal is bacterial cell wall extract.

Preferred methods include embodiments wherein said mimic of an injury signal is zymosan.

Preferred methods include embodiments wherein said mimic of an injury signal is interferon gamma.

Preferred methods include embodiments wherein said conditioned media is administered from allogeneic cells.

Preferred methods include embodiments wherein said conditioned media is administered from xenogeneic cells.

Preferred methods include embodiments wherein said conditioned media is administered from autologous cells.

Preferred methods include embodiments wherein said regenerative cells are exposed to hypoxia.

Preferred methods include embodiments wherein said regenerative cells are exposed to osmotic pressure.

Preferred methods include embodiments wherein said regenerative cells are exposed to hyperthermia.

Preferred methods include embodiments in contact with a liquid media, wherein said liquid media is selected from a group of media useful for maintaining cell viability in culture consisting of a group comprising of: a) alpha MEM; b) DMEM; c) RPMI; d) Opti-MEM; e) IMEM; and f) AIM-V media.

Preferred methods include embodiments in contact with a liquid media, wherein said cells are expanded in liquid media containing fetal calf serum and subsequently cultured in media substantially lacking said fetal calf serum, with said culture lacking fetal calf serum used for production of a therapeutic product.

Preferred methods include embodiments wherein cell cells are in contact with a liquid media of claim 1, wherein said contact between said cell and liquid media is between 1 minute to 96 hours.

Preferred methods include embodiments wherein said cells are in contact with a liquid media of claim 1, wherein said contact between said cell and liquid media is between 12 hours to 72 hours.

Preferred methods include embodiments wherein said cells are in contact with a liquid media of claim 1, wherein said contact between said cell and liquid media is between 24 hours to 48 hours.

Preferred methods include embodiments wherein said cells are in contact with a liquid media of claim 1, wherein said contact between said cell and liquid media is approximately 24 hours.

Preferred methods include embodiments wherein said cells are in contact with liquid media is selected for a timepoint in which optimal secretion of therapeutic factors occurs in said liquid media.

Preferred methods include embodiments wherein said therapeutic property endowed to said liquid media is ability to inhibit, alleviate, or resolve Orthopox Virus (Monkeypox, Smallpox, etc) infection.

Preferred methods include embodiments wherein said liquid media is combined with an immune stimulant.

Preferred methods include embodiments wherein said immune stimulant is interferon alpha.

Preferred methods include embodiments wherein said liquid media is concentrated and used for the formulation of a pharmaceutical.

Preferred methods include embodiments wherein said formulation generated from said liquid media is administered therapeutically from a group of routes of administration selected from: a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intra-nodally; x) by any parenteral route; and y) via inhalation

Preferred methods include embodiments wherein said cell population is immortalized.

Preferred methods include embodiments wherein said cell population is immortalized by means selected from a group comprising of: a) transfection with an oncogene; b) transfection telomerase; and c) transfection with a combination of an oncogene and telomerase.

Preferred methods include embodiments wherein said cell population is immortalized by means of transfection with an oncogene selected from a group of oncogenes comprising of: a) abl; b) Af4/hrx; c) akt-2; d) alk; e) alk/npm; f) aml1; g) aml1/mtg8; h) bcl-2, 3, 6; i) bcr/abl; j) c-myc; k) dbl; l) dek/can; m) E2A/pbx1; n) egfr; o) enl/hrx; p) erg/TLS; q) erbB; r) erbB-2; s) ets-1; t) ews/fli-1; u) fms; v) fos; w) fps; x) gli; y) gsp; z) gsp; aa) HER2/new; ab) hox11; ac) hst; ad) IL-3; ae) int-2; af) jun; ag) kit; ah) KS3; ai) K-sam; aj) Lbc; ak) 1ck; al) Imo1, Imo-2; am) L-myc; an) lyl-1; ao) lyt-10; ap)lyt-10/C alpha 1; aq) mas; ar) mdm-2; as) ml1; at) mos; au) mtg8/aml1; av) myb; aw) MYH11; ax) new; ay) N-myc; az) ost; ba) pax-5; bb) pbx1/E2a; bc) pim-1; bd) PRAD-1; be) raf; bf) RAR/PML; bg) Ras H, K, N; bh) rel/nrg; bi) ret; bj) rhom1, rhom2; bk) ros; bl) ski; bm) sis; bn) set/can; bo) src; bp) Tal1, tal1; bq) tan-1; br) Tiam1; bs) TSC2; and bt) trk.

Preferred methods include embodiments wherein regenerative cells themselves are administered for prevention of Monkeypox.

Preferred methods include embodiments wherein regenerative cells themselves are administered for treatment of Orthopox Virus (Monkeypox, Smallpox, etc) infection.

Preferred methods include embodiments wherein regenerative cell derived exosomes are administered for prevention of Orthopox Virus (Monkeypox, Smallpox, etc) infection.

Preferred methods include embodiments wherein regenerative cells derived exosomes are administered for treatment of Orthopox Virus (Monkeypox, Smallpox, etc) infection.

Preferred methods include embodiment of treating Orthopox Virus (Monkeypox, Smallpox, etc) infection comprising: a) selecting a patient suffering from Orthopox Virus (Monkeypox, Smallpox, etc) infection; and b) administering a regenerative cell population.

Preferred methods include embodiments wherein said regenerative cell population is transfected to express one or more cytokines with antiviral activity.

Preferred methods include embodiments wherein said regenerative cell population is transfected to express one or more cytokines with immune modulatory activity.

Preferred methods include embodiments wherein said regenerative cell population is transfected to express interferon alpha.

Preferred methods include embodiments wherein said regenerative cell population is transfected to express interferon beta.

Preferred methods include embodiments wherein said regenerative cell population is transfected to express interferon gamma.

Preferred methods include embodiments wherein said regenerative cell population is transfected to express interferon omega.

Preferred methods include embodiments wherein said regenerative cell population is transfected to express interferon tau.

Preferred methods include embodiments wherein said regenerative cell population is transfected to express TNF-alpha.

Preferred methods include embodiments wherein said regenerative cell population is transfected to express TNF-beta.

Preferred methods include embodiments wherein said regenerative cell population is transfected to express soluble RIG-1.

Preferred methods include embodiments wherein said regenerative cell population is transfected to express HMGB1.

Preferred methods include embodiments wherein said regenerative cell population is transfected to express G-CSF.

Preferred methods include embodiments wherein said regenerative cell population is transfected to express GM-CSF.

Preferred methods include embodiments wherein said regenerative cell population is transfected to express M-CSF.

Preferred methods include embodiments wherein said regenerative cell population is transfected to express interleukin 1.

Preferred methods include embodiments wherein said regenerative cell population is transfected to express interleukin 2.

Preferred methods include embodiments wherein said regenerative cell population is transfected to express interleukin 7.

Preferred methods include embodiments wherein said regenerative cell population is transfected to express interleukin 9.

Preferred methods include embodiments wherein said regenerative cell population is transfected to express interleukin 11.

Preferred methods include embodiments wherein said regenerative cell population is transfected to express interleukin 12 p35.

Preferred methods include embodiments wherein said regenerative cell population is transfected to express interleukin 12 p40.

Preferred methods include embodiments wherein said regenerative cell population is transfected to express interleukin 12 p35 and p40 heterodimer.

Preferred methods include embodiments wherein said regenerative cell population is transfected to express interleukin 15.

Preferred methods include embodiments wherein said regenerative cell population is transfected to express interleukin 18.

Preferred methods include embodiments wherein said regenerative cell population is transfected to express interleukin 17.

Preferred methods include embodiments wherein said regenerative cell population is transfected to express interleukin 20.

Preferred methods include embodiments wherein said regenerative cell population is transfected to express interleukin 23.

Preferred methods include embodiments wherein said regenerative cell population is transfected to express interleukin 27.

Preferred methods include embodiments wherein said regenerative cell population is transfected to express interleukin 33.

Preferred methods include embodiments wherein said regenerative cell population is transfected to express CD80.

Preferred methods include embodiments wherein said regenerative cell population is transfected to express CD86.

Preferred methods include embodiments wherein said regenerative cell population is transfected to express CD40.

Preferred methods include embodiments wherein said regenerative cell population is transfected to express siRNA targeting interleukin-10.

Preferred methods include embodiments wherein said regenerative cell population is transfected to express siRNA targeting leukemia inhibitory factor.

Preferred methods include embodiments wherein said regenerative cell population is transfected to express siRNA targeting TGF-alpha.

Preferred methods include embodiments wherein said regenerative cell population is transfected to express siRNA targeting TGF-beta.

Preferred methods include embodiments wherein said regenerative cell population is transfected to express siRNA targeting HLA-G.

Preferred methods include embodiments wherein said regenerative cell population is transfected to express siRNA targeting interleukin 4.

Preferred methods include embodiments wherein said regenerative cell population is transfected to express siRNA targeting interleukin 6.

Preferred methods include embodiments wherein said regenerative cell population is transfected to express siRNA targeting interleukin 13.

Preferred methods include embodiments wherein said regenerative cell population is transfected to express siRNA targeting interleukin 35.

Preferred embodiments include methods of preventing and/or treating Orthopox Virus (Monkeypox, Smallpox, etc) infection wherein a cell based pouch is implanted encapsulated in a manner so as to allow for release of exosomes, soluble factors, and microvesicles from said regenerative cells.

Preferred methods include embodiments wherein said regenerative cell is a mesenchymal stem cell that is derived from a group of sources comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of: a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and d) CD45.

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A, -B, -C.

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR, -DP, -DQ.

Preferred methods include embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1.

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said umbilical cord tissue-derived cells are capable of differentiating into one or more lineages selected from a group comprising of; a) ectoderm; b) mesoderm, and; c) endoderm.

Preferred methods include embodiments wherein said bone marrow derived mesenchymal stem cells possess markers selected from a group comprising of: a) CD73; b) CD90; and c) CD105.

Preferred methods include embodiments wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Preferred methods include embodiments wherein said bone marrow derived mesenchymal stem cell is a mesenchymal stem cell progenitor cell.

Preferred methods include embodiments wherein said mesenchymal progenitor cells are a population of bone marrow mesenchymal stem cells enriched for cells containing STRO-1.

Preferred methods include embodiments wherein said mesenchymal progenitor cells express both STRO-1 and VCAM-1.

Preferred embodiments include methods wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67; and k) glycophorin A.

Preferred methods include embodiments wherein said bone marrow mesenchymal stem cells lack expression of CD14, CD34, and CD45.

Preferred methods include embodiments wherein said STRO-1 expressing cells are positive for a marker selected from a group comprising of: a) VACM-1; b) TKY-1; c) CD146 and; d) STRO-2.

Preferred methods include embodiments wherein said bone marrow mesenchymal stem cell express markers selected from a group comprising of: a) CD13; b) CD34; c) CD56 and; d) CD117.

Preferred methods include embodiments wherein said bone marrow mesenchymal stem cells do not express CD10.

Preferred methods include embodiments wherein said bone marrow mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45, or CD64.

Preferred methods include embodiments wherein said skeletal muscle stem cells express markers selected from a group comprising of: a) CD13; b) CD34; c) CD56 and; d) CD117.

Preferred methods include embodiments wherein said skeletal muscle mesenchymal stem cells do not express CD10.

Preferred methods include embodiments wherein said skeletal muscle mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 or CD64.

Preferred methods include embodiments wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Preferred methods include embodiments wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Preferred methods include embodiments wherein said subepithelial umbilical cord derived mesenchymal stem cells express CD29, CD73, CD90, CD166, SSEA4, CD9, CD44, CD146, and CD105.

Preferred methods include embodiments wherein said subepithelial umbilical cord derived mesenchymal stem cells do not express CD45, CD34, CD14, CD79, CD106, CD86, CD80, CD19, CD117, Stro-1, and HLA-DR.

Preferred methods include embodiments wherein said subepithelial umbilical cord derived mesenchymal stem cells are positive for S OX2.

Preferred methods include embodiments wherein said subepithelial umbilical cord derived mesenchymal stem cells are positive for OCT4.

Preferred methods include embodiments wherein said subepithelial umbilical cord derived mesenchymal stem cells are positive for OCT4 and SOX2.

Preferred embodiments include methods of treating Orthopox Virus (Monkeypox, Smallpox, etc) infection comprising administration of regenerative cell derived exosomes.

Preferred methods include embodiments wherein said exosomes express CD9.

Preferred methods include embodiments wherein said exosomes express annexin-V.

Preferred methods include embodiments wherein said exosomes express LFA-1.

Preferred methods include embodiments wherein said exosomes express CD73.

Preferred methods include embodiments wherein said exosomes express LAP.

Preferred methods include embodiments wherein said exosomes express mR-155.

Preferred methods include embodiments wherein said exosomes express mR-287.

Preferred methods include embodiments wherein said exosomes express a tetraspanin molecule.

Preferred methods include embodiments wherein said exosomes express HLA-G.

Preferred methods include embodiments wherein said exosomes express ILT-3

Preferred methods include embodiments wherein said exosomes express ILT-4.

Preferred methods include embodiments wherein said exosomes express sialomucin.

Preferred embodiments include methods of treating Orthopox Virus (Monkeypox, Smallpox, etc) infection comprising administration of regenerative cell derived smart nanoexosomes.

Preferred methods include embodiments wherein said smart nanoexosomes are derived from mesenchymal cells from recently infected patients.

Preferred methods include embodiments wherein said smart nanoexosomes express LAMP

Preferred methods include embodiments wherein said smart nanoexosomes express CD63

### DETAILED DESCRIPTION OF THE INVENTION

The invention teaches methods of generating an Orthopox Virus (Monkeypox, Smallpox, etc) prophylactic and/or therapeutic product through growth of various cell populations in a liquid media and extraction of said liquid media. In some manifestations the invention describes utilization of MSC conditioned media for prophylaxis and/or therapy of Monkeypox. In other embodiments the invention provides means of stimulating MSC before collection of conditioned media.

As used herein and in the appended claims, the singular forms “a,” “and,” and “the” include plural referents unless the context clearly dictates otherwise. Thus, for example, reference to “a cell” includes a plurality of such cells and reference to “the agent” includes reference to one or more agents known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings/

“Biocompatible polymers” used in the present disclosure are selected from the group consisting of carbomers (acrylic acid polymers crosslinked with a polyalkenyl polyether), polyalkylene glycols (for example, polyethylene glycols and polypropylene glycols), poloxamers (polyoxyethylene-polyoxypropylene block copolymers), polyesters, polyethers, polyanhydrides, polyacrylates, polyvinyl acetates, polyvinyl pyrrolidones, and polysaccharides such as, for example, hyaluronic acid, derivatives of hyaluronic acid, in particular crosslinked hyaluronic acid and esters of hyaluronic acid (for example, benzyl ester of hyaluronic acid), hydroxyalkylcelluloses (for example, hydroxymethylcellulose and hydroxyethylcellulose), and carboxyalkylcelluloses (for example, carboxymethylcellulose).

“Growth factor” refers to any material or materials having a positive reaction on living tissues, such as promoting the growth of tissues. Exemplary growth factors include, but are not limited to, platelet-derived growth factor (PDGF), platelet-derived angiogenesis factor (PDAF), vascular endothelial growth factor (VEGF), platelet-derived epidermal growth factor (PDEGF), platelet factor 4 (PF-4), transforming growth factor beta. (TGF-B), acidic fibroblast growth factor (FGF-A), basic fibroblast growth factor (FGF-B), transforming growth factor A (TGF-A), insulin-like growth factors 1 and 2 (IGF-1 and IGF-2), B thromboglobulin-related proteins (BTG), thrombospondin (TSP), fibronectin, von Wallinbrand's factor (vWF), fibropeptide A, fibrinogen, albumin, plasminogen activator inhibitor 1 (PAI-1), osteonectin, regulated upon activation normal T cell expressed and presumably secreted (RANTES), gro-A, vitronectin, fibrin D-dimer, factor V, antithrombin III, immunoglobulin-G (IgG), immunoglobulin-M (IgM), immunoglobulin-A (IgA), a2-macroglobulin, angiogenin, Fg-D, elastase, keratinocyte growth factor (KGF), epidermal growth factor (EGF), fibroblast growth factor (FGF), tumor necrosis factor (TNF), fibroblast growth factor (FGF) and interleukin-1 (IL-1), Keratinocyte Growth Factor-2 (KGF-2), and combinations thereof. One of the important characteristics common to the above listed growth factors is that each substance is known or believed to have a positive reaction on living tissue, known as bioactivity, to enhance cell or tissue growth.

The term “mesenchymal stem cell” refers to but in no way is limited to, those described in the following references, the disclosures of which are incorporated herein by reference: U.S. Pat. Nos. 5,215,927; 5,225,353; 5,262,334; 5,240,856; 5,486,359; 5,759,793; 5,827,735; 5,811,094; 5,736,396; 5,837,539; 5,837,670; 5,827,740; 6,087,113; 6,387,367; 7,060,494; 8,790,638; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

In one embodiment, the invention provides a means of creating a medicament useful for the treatment of Monkeypox virus through culturing Wharton Jelly mesenchymal cells in a serum free media. Many types of media may be used and chosen by one of skill in the art. In one embodiment a media is selected from a group comprising of alpha MEM, DMEM, RPMI, Opti-MEM, IMEM, and AIM-V. Cells may be cultured in a variety of media for expansion that contain fetal calf serum, or other growth factors, however, for collection of therapeutic supernatant, in a preferred embodiment, the cells are transferred to a media substantially lacking serum. In some embodiments, the supernatant is administered directly into the patient in need of treatment. It is well known in the art that preparation of the supernatant before administration may be performed by various means, for example, said supernatant may be filter sterilized, or in some conditions concentrated. In a preferred embodiment, the supernatant is administrated intramuscularly in a volume of 0.5 to 1 ml per injection, with two injections per week. In this embodiment, a concentration of 30 million Wharton Jelly mesenchymal cells are grown on a plastic surface for approximately 24 hours. Supernatant is harvested, filter sterilized, and stored for administration.

In one aspect of the invention, potency of the conditioned media product may be quantified by use of assessing protein production. Such assays are well-known to one skilled in the art. For quantification of anti-inflammatory activity, the term “inflammation” will be understood by those skilled in the art to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this invention, inflammatory pain and/or fever caused by inflammation.

In another embodiment, conditioned media is generated in an ex-vivo extracorporal setting. Specifically, cells of interest are grown on the outside of a hollow-fiber filter which is connected to a continuous extracorporeal system. Said hollow-fiber system contains pores in the hollow fiber of sufficient size so has to allow exchange of proteins between circulating blood cells and cultured cells on the outside of the hollow fibers, without interchange of host cells with said stem cells.

In one embodiment, stem cell conditioned media is used in combination with an immune suppressive agent to augment its activity. In some cases, viral infections media pathology through immune hyperactivation. While stem cell conditioned media may be used alone for treatment and/or maintenance of disease remission, in some embodiments coadministration with an immune suppressive agent may be required. Additionally, an immune suppressive agent may be useful for “induction therapy”. Depending on disease and response desired, it will be known to one of skill in the art to choose from various immune suppressive agents. For example, some immune suppressive agents, such as anti-CD52 antibodies may be used when a systemic depletion of T and B cells is desired, whereas agents that concurrently stimulate T regulatory cell activity, such as Rapamycin, may be desired in other cases. The skilled practitioner is guided to several agents that are known in the art for causing immune suppression, which include cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex.®., and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc.

In another embodiment, the use of stem cell conditioned media may be used to potentiate an existing anti-inflammatory agent for use in Monkeypox prophylaxis and/or therapy. Anti-inflammatory agents may comprise one or more agents including NSAIDs, interleukin-1 antagonists, dihydroorotate synthase inhibitors, p38 MAP kinase inhibitors, TNF-.alpha. inhibitors, TNF-.alpha. sequestration agents, and methotrexate. More specifically, anti-inflammatory agents may comprise one or more of, e.g., anti-TNF-.alpha., lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, etc.

In one embodiment of the invention, conditioned media is administered together with an inhibitor of NF-kappa B. A variety of inhibitors are known in the art and may be selected from a list comprising of: NF-kappa B inhibitor is selected from a group of compounds comprising of: inhibitor of NF-kappa B is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

In other embodiments, the conditioned media is used as an active ingredient for the generation of a pharmaceutical formulation for the prophylaxis and/or treatment of Orthopox Virus (Monkeypox, Smallpox, etc) infection. This may comprise administration of the stem cell conditioned media therapeutic agent alone, but preferably comprise administration by way of known pharmaceutical formulations, including tablets, capsules or elixirs for oral administration, suppositories for rectal administration, sterile solutions or suspensions for parenteral or intramuscular administration, liposomal or encapsulated formulations, formulations wherein the therapeutic agent is alone or conjugated to a delivery agent or vehicle, and the like. It will be appreciated that therapeutic entities of the invention will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15. sup. th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin.™.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998) and the citations therein for additional information related to excipients and carriers well known to pharmaceutical chemists. In one embodiment of the invention, one or more agents of the invention are nanoencapsulated into nanoparticles for delivery. The nanoencapsulation material may be biodegradable or nondegradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Sawhney et al., Macromolecules 26:581-587 (1993). Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic.™. surfactant (BASF). Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen. These polymers are merely exemplary of the types of nanoencapsulation materials that can be utilized and are not intended to represent all the nanoencapsulation materials within which entrapment is possible. In one embodiment, the therapeutic agent is administered in a topical formulation. Topical formulations are useful in the treatment of conditions associated with dermal diseases. For example, topical administration of stem cell conditioned media may be performed for the treatment of psoriasis, scleroderma, or acne. Topical forms of administration may consist of, for example, aqueous and nonaqueous gels, creams, multiple emulsions, microemulsions, liposomes, ointments, aqueous and nonaqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

In one embodiment, the pharmaceutically acceptable carrier is a liposome or a transdermal enhancer. Topical formulations of the invention may include a dermatologically acceptable carrier, e.g., a substance that is capable of delivering the other components of the formulation to the skin with acceptable application or absorption of those components by the skin. The carrier will typically include a solvent to dissolve or disperse the therapeutic agent, and, optionally one or more excipients or other vehicle ingredients. Carriers useful in accordance with the topical formulations of the present invention may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the invention are prepared by mixing a compound of the invention with a topical carrier according to well-known methods in the art, for example, methods provided by standard reference texts e.g., Remington: The Science and Practice of Pharmacy, 1577-1591, 1672-1673, 866-885 (Alfonso R. Gennaro ed. 19th ed. 1995); and Ghosh et al., Transdermal and Topical Drug Delivery Systems (1997). In other embodiments, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations of the invention if desired. A topical formulation of the invention may be designed to be left on the skin and not washed shortly after application. Alternatively, the topical formulation may be designed to be rinsed off within a given amount of time after application.

For use in the context of the present invention, pluripotent stem cells possess certain desirable properties, which include unique “early” growth factor production profile and possessing antiviral/immune stimulating activity relevant to Monkeypox virus prophylaxis and/or therapy. It is believed in the art that many of the therapeutic effects of ES cell administration are mediated by paracrine factors. This is promising since therapeutic use of ES cells themselves is limited by formation of teratoma. Another embodiment of the current invention is the use of embryonic stem cell supernatant as a therapeutic product. Specific embodiments include identification of substantially purified fractions of said supernatant capable of inducing endothelial cell proliferation, smooth muscle regeneration, and/or neuronal cell proliferation/survival, and/or anti-inflammatory activity, and/or stimulation of endogenous reparative processes. Identification of such therapeutically active fractions may be performed using methods commonly known to one skilled in the art, and includes separation by molecular weight, charge, affinity towards substrates and other physico-chemical properties. In one particular embodiment, supernatant of embryonic stem cell cultures is harvested substantially free from cellular contamination by use of centrifugation or filtration. Supernatant may be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of embryonic stem cell supernatant may be passed through each cartridge before elution. After washing the cartridges, material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the embryonic stem cell culture supernatant, and allows for the elimination of salts and other polar contaminants. It may, however, be desired to use other adsorption means in order to purify certain compounds from the embryonic stem cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5. times. 54 cm glass column and equilibrated with 3 column volumes of the same buffer. Embryonic stem cell supernatant concentrates extracted by C18 cartridge may be dissolved in 0.5 ml of 20 mM Tris buffer, pH 7.2, and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically. For the practice of the invention, the practitioner is referred to the numerous methods of generating embryonic stem cells that are known in the art. Patents describing the generation of embryonic stem cells include U.S. Pat. No. 6,506,574 to Rambhatla, U.S. Pat. No. 6,200,806 to Thomson, U.S. Pat. No. 6,432,711 to Dinsmore, and U.S. Pat. No. 5,670,372 to Hogan.

For the practice of the invention, supernatants generated by culture with cells may be administered to the patient in an injection solution, which may be saline, mixtures of autologous plasma together with saline, or various concentrations of albumin with saline. Ideally, the pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. Injection can be performed systemically, or more specifically, via routes of administration selected from: a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intranodally; x) by any parenteral route; and y) via inhalation.

Immune modulatory stem cells may be derived from other stem cell sources besides MSC, however, in the case of MSC, said cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Further embodiments encompass methods wherein mesenchymal stem cells do not express substantial levels of HLA-DR, CD117, and CD45. Further embodiments encompass methods wherein mesenchymal stem cells are derived from a group selected from: bone marrow, adipose tissue, umbilical cord blood, placental tissue, peripheral blood mononuclear cells, differentiated embryonic stem cells, and differentiated progenitor cells. Further embodiments encompass methods wherein germinal stem cells express markers selected from a group comprising of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1. Further embodiments encompass methods wherein adipose tissue derived stem cells express markers are selected from a group consisting of: CD13, CD29, CD44, CD63, CD73, CD90, CD166, Aldehyde dehydrogenase (ALDH), and ABCG2. Further embodiments encompass methods wherein adipose tissue derived stem cells are a population of purified mononuclear cells extracted from adipose tissue capable of proliferating in culture for more than 1 month. Further embodiments encompass methods wherein exfoliated teeth derived stem cells express markers selected from the group consisting of: STRO-1, CD146 (MUC18), alkaline phosphatase, MEPE, and bFGF. Further embodiments encompass methods wherein hair follicle stem cells express markers selected from a group comprising of: cytokeratin 15, Nanog, and Oct-4. Further embodiments encompass methods wherein hair follicle stem cells are capable of proliferating in culture for a period of at least one month. Further embodiments encompass methods wherein hair follicle stem cells secrete one or more of the following proteins when grown in culture: basic fibroblast growth factor (bFGF), endothelin-1 (ET-1) and stem cell factor (SCF). Further embodiments encompass methods wherein dermal stem cells express markers selected from a group comprising of: CD44, CD13, CD29, CD90, and CD105. Further embodiments encompass methods wherein dermal stem cells are capable of proliferating in culture for a period of at least one month. Further embodiments encompass methods wherein parthenogenically derived stem cells are generated by addition of a calcium flux inducing agent to activate an oocyte followed by enrichment of cells expressing markers selected from a group comprising of SSEA-4, TRA 1-60 and TRA 1-81. Further embodiments encompass methods wherein reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions. Further embodiments encompass methods wherein nuclear transfer comprises introducing nuclear material to a cell substantially enucleated, said nuclear material deriving from a host whose genetic profile is sought to be dedifferentiated. Further embodiments encompass methods wherein cytoplasmic transfer comprises introducing cytoplasm of a cell with a dedifferentiated phenotype into a cell with a differentiated phenotype, such that said cell with a differentiated phenotype substantially reverts to a dedifferentiated phenotype. Further embodiments encompass methods wherein DNA demethylating agent is selected from a group comprising of: 5-azacytidine, psammaplin A, and zebularine. Further embodiments encompass methods wherein histone deacetylase inhibitor is selected from a group comprising of: valproic acid, trichostatin-A, trapoxin A and depsipeptide. Further embodiments encompass methods wherein cells are identified based on expression multidrug resistance transport protein (ABCG2) or ability to efflux intracellular dyes such as rhodamine-123 and or Hoechst 33342. Further embodiments encompass methods wherein cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In some embodiments, said endocrine regenerative unit generated from coculture of mesenchymal stem cells, or other stem cells, together with immature dendritic cells, and/or cytokines and growth factors are utilized as in vivo factories of immune-modulatory exosomes. Alternatively, tolerogenic exosomes generated from said immune modulatory units maybe collected ex vivo and subsequently utilized for induction of antigen specific tolerance.

Exosomes are nanoparticles (40-100 nm) in size that possess highly defined homogeneous characteristics [1]. Originally thought to be a by-product of cell protein turnover [2], these particles are becoming appreciated as a critical means of intracellular communication in areas ranging from neurotransmission [3], to immune modulation [2], to infectious disease [4]. Compared with other secreted vesicles, exosomes have much better defined biophysical and biochemical properties, specifically, they have a diameter of 40-100 nm (with a density in sucrose of 1.13-1.19 g/ml), and can be sedimented at 100,000 g [1]. Their membranes are enriched in cholesterol, sphingomyelin and ceramide, and are known to contain lipid rafts. Exosomes were originally discovered as a means of exportation of the transferin receptor during sheep reticulocyte maturation [5]. In recent years, an explosion of interest in exosomes has occurred, with a wide variety of cells being reported to secrete these nanoparticles ranging from T cells [6, 7], B cells [8, 9], dendritic cells [10, 11], tumor cells [12, 13], neurons [14, 15], oligodendrocytes [16], and placental cells [17]. It is believed that exosomes play fundamental role in immune escape of the “fetal allograft” [18]. Endometrial regenerative cells (ERC) are believed to be precursors of MSC that development into the maternal portion of the placenta. Given the high angiogenic activity of ERC, as well as their ability to induce therapeutic effects in xenogeneic immune competent models [19], one embodiment of the invention is the utilization of ERC exosomes as a non-cellular method of inducing immune modulatory effects that are present when ERC are administered therapeutically. In another embodiment, exosomes from other stem cells may be utilized for immunotherapeutic purposes.

Immunological functions of exosomes were first identified in B cells [20], through studies demonstrating that these cells contain a late endocytic compartment, called MIIC (major histocompatibility complex [MHC] class II-enriched compartment), that harbors newly synthesized MHC class II molecules in transit to the plasma membrane. It was found that the MIIC compartment would fuse with the plasma membrane, but instead of the MHC II molecules becoming membrane bound, some would be found in the soluble fraction. These particles, which the investigators termed “exosomes” in reference to original work on reticulocytes [5], were demonstrated to possess a distinct surface composition as compared to the plasma membrane. Interestingly, in the exosomes, a high concentration of MHC I and II, as well as antigen were found.

In 2004, our group filed Canadian Patent #2453198 entitled “QUANTIFICATION AND GENERATION OF IMMUNE SUPPRESSIVE EXOSOMES”. To our knowledge these were the first data demonstrating that in certain contexts, exosomes may suppress the immune system. This data, which were subsequently published, demonstrated that exosomes from prostate cancer patients suppress T cell activation in an MHC I and Fas ligand dependent manner [21]. In one embodiment of the invention, methodologies used for purification of immune suppressive exosomes from tumor cells, incorporated by reference, are applied to conditioned media of stem cells, specifically of mesenchymal stem cells, and more specifically of endometrial regenerative cells, in order to isolate, concentrate and therapeutically administer exosomes derived from stem cells for immune modulatory purposes.

Smart nanoexosomes are endogenous bubble vesicles that are formed by germination in endosome division during endosome maturation, from primary to secondary endosomes in the form of multi-vesicular bodies. The smart nanoexosomes are generated by a process of infiltration into endosomal membranes to form molecular vesicles. The formation of nanoexosomes begins with germination inside the endosomal membrane to form nanoexosomes vesicles in the cytoplasm. A complex endosomal assembly required for the carrier vesicles or ceramide sphingolipids. The formation of nanoexosomes vesicles during their formation shows some similarities with the exosome vesicles formed during lysosome formation, including the surface proteins of lysosomes, which are also present in exosomal membranes. Numerous external factors, including exocytosis, cell type, the presence or absence of cytokines, serum conditions and growth factors, affect the biogenesis of nanoexosomes. In addition, protein sorting, trans-acting mediators, nanoexosome sites and physical and chemical aspects regulate biogenesis. In one embodiment of the invention, smart nanoexosomes are generated from the tissue mesenchymal cells from recently infected patients with Monkeypox. The smart nanoexosomes have the ability to attenuate the inflammatory response and is beneficial to new patients who have been infected. Invitro, the smart nanoexosomes attenuate the infectiveness of the Monkeypox.

### References

1. Thery, C., M. Ostrowski, and E. Segura, Membrane vesicles as conveyors of immune responses. Nature reviews. Immunology, 2009. 9(8): p. 581-93.
2. Ludwig, A. K. and B. Giebel, Exosomes: Small vesicles participating in intercellular communication. The international journal of biochemistry & cell biology, 2011.
3. Alvarez-Erviti, L., et al., Lysosomal dysfunction increases exosome-mediated alpha-synuclein release and transmission. Neurobiology of disease, 2011. 42(3): p. 360-7.
4. Silverman, J. M. and N. E. Reiner, Exosomes and other microvesicles in infection biology: organelles with unanticipated phenotypes. Cellular microbiology, 2011. 13(1): p. 1-9.
5. Pan, B. T. and R. M. Johnstone, Fate of the transferrin receptor during maturation of sheep reticulocytes in vitro: selective externalization of the receptor. Cell, 1983. 33(3): p. 967-78.
6. Alonso, R., et al., Diacylglycerol kinase alpha regulates the formation and polarisation of mature multivesicular bodies involved in the secretion of Fas ligand-containing exosomes in T lymphocytes. Cell death and differentiation, 2011. 18(7): p. 1161-73.
7. Zhang, H., et al., CD4(+) T cell-released exosomes inhibit CD8(+) cytotoxic T-lymphocyte responses and antitumor immunity. Cellular & molecular immunology, 2011. 8(1): p. 23-30.
8. Mathews, J. A., et al., CD23 Sheddase A disintegrin and metalloproteinase 10 (ADAM10) is also required for CD23 sorting into B cell-derived exosomes. The Journal of biological chemistry, 2010. 285(48): p. 37531-41.
9. Buschow, S. I., et al., MHC class II-associated proteins in B-cell exosomes and potential functional implications for exosome biogenesis. Immunology and cell biology, 2010. 88(8): p. 851-6.
10. Hwang, I. and D. Ki, Receptor-mediated T cell absorption of antigen presenting cell-derived molecules. Frontiers in bioscience : a journal and virtual library, 2011. 16: p. 411-21.
11. Viaud, S., et al., Updated technology to produce highly immunogenic dendritic cell-derived exosomes of clinical grade: a critical role of interferon-gamma. Journal of immunotherapy, 2011. 34(1): p. 65-75.
12. Clayton, A., et al., Cancer exosomes express CD39 and CD73, which suppress T cells through adenosine production. Journal of immunology, 2011. 187(2): p. 676-83.
13. Battke, C., et al., Tumour exosomes inhibit binding of tumour-reactive antibodies to tumour cells and reduce ADCC. Cancer immunology, immunotherapy : CII, 2011. 60(5): p. 639-48.
14. Lachenal, G., et al., Release of exosomes from differentiated neurons and its regulation by synaptic glutamatergic activity. Molecular and cellular neurosciences, 2011. 46(2): p. 409-18.
15. Faure, J., et al., Exosomes are released by cultured cortical neurones. Molecular and cellular neurosciences, 2006. 31(4): p. 642-8.
16. Fitzner, D., et al., Selective transfer of exosomes from oligodendrocytes to microglia by macropinocytosis. Journal of cell science, 2011. 124(Pt 3): p. 447-58.
17. Mincheva-Nilsson, L. and V. Baranov, The role of placental exosomes in reproduction. American journal of reproductive immunology, 2010. 63(6): p. 520-33.
18. Mincheva-Nilsson, L., et al., Placenta-derived soluble MHC class I chain-related molecules down-regulate NKG2D receptor on peripheral blood mononuclear cells during human pregnancy: a possible novel immune escape mechanism for fetal survival. Journal of immunology, 2006. 176(6): p. 3585-92.
19. Murphy, M. P., et al., Allogeneic endometrial regenerative cells: an “Off the shelf solution” for critical limb ischemia? Journal of translational medicine, 2008. 6: p. 45.
20. Raposo, G., et al., B lymphocytes secrete antigen-presenting vesicles. The Journal of experimental medicine, 1996. 183(3): p. 1161-72.
21. Abusamra, A. J., et al., Tumor exosomes expressing Fas ligand mediate CD8+ T-cell apoptosis. Blood cells, molecules & diseases, 2005. 35(2): p. 169-73.

1. A method for prophylaxis and/or treatment of Orthopox viruses to a patient in need comprising: a) obtaining a regenerative cell population; b) stimulating said regenerative cell population with a ligand for Toll-Like Receptors (TLRs) by itself or with a receptor selected from the group consisting of a NOD-Like Receptors (NLRs), and a RIG-I-Like Receptors (RLRs); c) collecting supernatant from said stimulated cells; d) concentrating active ingredients from said supernatant; and e) administering said concentrated supernatant to the patient in need.

2. The method of claim 1, wherein said regenerative cell population is a mesenchymal stem cell (MSC).

3. The method of claim 2, wherein said MSC is derived from a source selected from the group consisting of: a) peripheral blood; b) bone marrow; c) placenta; d) umbilical cord blood; e) menstrual blood; f) amniotic fluid; g) cerebral spinal fluid; h) umbilical cord tissue; i) liver: j) spleen; k) kidney; l) skin; and m) adipose tissue.

4. The method of claim 2, wherein said MSC express VEGF-receptor 2.

5. The method of claim 1, wherein said cellular population is monocytes.

6. The method of claim 1, wherein said cellular population is thymic medullary epithelial cells.

7. The method of claim 1, wherein said cellular population is hematopoietic stem cells.

8. The method of claim 1, wherein said dedifferentiation is accomplished by introduction into cells proteins capable of inducing dedifferentiation.

9. The method of claim 8, wherein said regenerative cell is from dedifferentiation which results in cells expression pluripotency markers.

10. The method of claim 9, wherein said pluripotency marker is TRA-1-60.

11. The method of claim 1, wherein said conditioned media is administered from allogeneic cells.

12. The method of claim 1, wherein said regenerative cells are exposed to hypoxia.

13. The method of claim 1, wherein said therapeutic property endowed to said liquid media is ability to inhibit, alleviate, or resolve Orthopox Virus infection.

14. The method of claim 1, wherein said liquid media is combined with an immune stimulant that is interferon alpha. The method of claim 1, wherein said liquid media is concentrated and used for the formulation of a pharmaceutical.

16. The method of claim 1, wherein said formulation generated from said liquid media is administered therapeutically from a group of routes of administration selected from the group consisting of: a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intra-nodally; x) by any parenteral route; and y) via inhalation.

17. The method of claim 16 wherein regenerative cells themselves are administered for prevention and/or treatment of Orthopox Virus infection.

18. The method of claim 1, wherein exosomes are derived from the regenerative cells and are administered for prevention and/or treatment of Orthopox Virus infection.

19. A method of treating Orthopox Virus infection comprising administration of regenerative cell derived exosomes to a patient in need.

20. The method of claim 19, wherein said exosomes express mR-155.
